(19) United States
(12) Patent Application Publication
(10) Pub. No.: US 2021/0379215 A1
(43) Pub. Date: Dec. 9, 2021

(54) METHODS AND APPARATUS USING FAR-ULTRAVIOLET LIGHT TO INACTIVATE PATHOGENS

(71) Applicants: Brian Kelleher, Del Mar, CA (US); Coleman Owen, Del Mar, CA (US); Ron Lenk, Albuquerque, NM (US)

(72) Inventors: Brian Kelleher, Del Mar, CA (US); Coleman Owen, Del Mar, CA (US); Ron Lenk, Albuquerque, NM (US)

(21) Appl. No.: 16/891,095

(22) Filed: Jun. 3, 2020

(51) Int. Cl. A61L 2/00; A61L 2/10

(52) U.S. Cl. CPC A61L 2/0047; A61L 2101/02; A61L 2/10

(57) ABSTRACT

The present invention involves methods and apparatus for destroying or inactivating pathogens in body cavities or on body surfaces using ultraviolet (UV) light of various wavelengths. More particularly, the invention comprises a portable apparatus containing an energy source, UV light emitter, insertion portion and controller. The energy source may be a battery and some embodiments may include a power converter. The UV emitter may be an LED, excimer lamp, laser, laser diode, fluorescent lamp, xenon flash lamp, arc lamp or gas discharge lamp. The insertion portion may be configured to enter certain body cavities and to transmit the UV light to target areas on body surfaces or within body cavities. The device may also incorporate light filters to block unwanted wavelengths. Some embodiments include a light sensor to detect visible and UV light and may measure the intensity and spectral characteristics of the UV emitter. In some embodiments, the invention includes a cap that covers the light guide, and optionally includes means for self-disinfecting the light guide and cap. In other embodiments, the invention includes a barrier element to prevent UV light from escaping from the body cavities. In some embodiments, the invention includes a securement mechanism to hold the insertion portion of the device within the body cavities. Specific applications include disinfection of all body cavities including nasal and oral cavities and auditory canals, as well as skin infections, lacerations, ulcers, lesions, surgical wounds, nails and tooth pockets. Certain embodiments disclose methods for inactivating certain pathogens in body cavities without destroying the normal microbiome of such cavities.

### CROSS-REFERENCE TO RELATED APPLICATIONS

N/A.

### BACKGROUND

### Field of Invention

This invention relates to the field of disinfection and more particularly to methods and apparatus for destroying or inactivating pathogens within a mammalian body cavity or on a portion of a mammalian body surface.

### Background of the Invention

It is well known that many pathogens such as bacteria and viruses can invade and infect various mammalian body cavities and body surfaces as well as invade and infect any disruption or breakdown of the dermal barrier. In particular, it is known that viruses such as rhinovirus and coronavirus can accumulate in the nasopharyngeal passageways, where they attack host cells and replicate. Recent studies suggest that the SARS-CoV-2 virus (covid-19) may use the nasal epithelium as a portal for initial infection and transmission (Nature Medicine, April 2020, 26, 681-687). It is also well known that ultraviolet (UV) light can destroy bacteria and inactivate viruses (Public Health Rep. 2010 January-February; 125(1): 15-27).

Ultraviolet light can be segregated into four wavelength ranges. UVA is between 315 nm and 400 nm. UVB is between 280 nm and 315 nm. UVC is between 200 nm and 280 nm. Vacuum UV (VUV) is between 100 nm and 200 nm. Both UVA and UVB are effective in destroying certain bacteria and inactivating certain viruses, but both wavelength ranges are known to pose potential harm to the host, causing erythema, eye injury and in some cases cellular damage that can lead to skin cancer. UVC is even more effective at destroying and inactivating such pathogens but wavelengths between about 240 and 280 also cause erythema, eye injury and may lead to skin cancer.

Recent research has shown that wavelengths 207 nm and 222 nm are highly effective at destroying and inactivating pathogens including airborne coronaviruses (Buonanno et al, “Far-UVC light efficiently and safely inactivates airborne human coronaviruses” Nature Research, pre-pub manuscript) without inducing mammalian skin damage (Buonanno et al “Germicidal Efficacy and Mammalian Skin Safety of 222-nm UV Light” Radiat Res. 2017 April; 187(4):483-491) or eye injury (Kaidzu et al “Evaluation of Acute Corneal Damage Induced by 222-nm and 254-nm Ultraviolet Light in SpragueDawley Rats”, Free Radic Res. 2019 June; 53(6):611-617). The Buonanno reference states “We found that low doses of, respectively 1.7 and 1.2 mJ/cm2 inactivated 99.9% of aerosolized alpha coronavirus 229E and beta coronavirus OC43 . . . . As all human coronaviruses have similar genomic sizes which is a primary determinant of UV sensitivity, it is reasonable to expect that far-UVC light will show similar inactivation efficiency against all human coronaviruses, including SARS-CoV-2.” Therefore, although direct testing has yet to be completed on SARS-CoV-2, it is likely that exposure to far-UV with radiant exposure levels of about 1-2 mJ/cm2 will have similar inactivation rates for this virus as for other coronaviruses.

Various methods and apparatus have been developed for applying UV light to body cavities such as the nasopharyngeal passageways. For example, Gertner US20060167531 discloses a set of embodiments that comprise an energy source, light source and light guide adaptable for delivering UV light the nasopharyngeal passageways. Gertner discloses the use of UVA, B and C.

Several devices are commercially available to apply UV light to destroy or inactivate pathogens. The Rhinolight comprises a bulky external power supply and light source with a flexible conduit connected to an insertion portion. The system is intended for use by trained clinicians to apply 5% UV-B, 25% UV-A plus 70% visible light to the nasopharyngeal passageways in a series of treatments. The mode of action is to suppress the immune system and thus reduce the inflammatory response to allergens; it is not intended to inactivate pathogens.

Another commercially available device is the UV Aid product, which is handheld. This device comprises a battery, a control circuit, a UVA LED and a light guide. The method of use involves pressing an activation button and inserting the insertion portion into a nostril for a period of time. The instructions state that UV Aid may be also used to disinfect or sterilize other body parts such as the hands. The drawback of this device is that it relies on UVA as the mode of action, and UVA is known to cause erythema and potential eye injury as well as cellular damage that may lead to skin cancer. It is also less effective at destroying and inactivating certain pathogens than UVC. Further, the UV Aid does not include features to prevent over-exposure or eye injury.

Although UV light, and particularly UVC light, is highly capable of destroying or inactivating pathogens on the skin or in body cavities, there is reason to use restraint in the application of such therapy. Even if the far-UV wavelengths between 200 nm and 230 nm prove to be as safe as has been recently reported, the use of this light in high doses has the risk of not only killing or inactivating the target pathogens but also disrupting or eliminating the normal microbiome of the body cavity. Several studies have noted the importance of preserving the normal microbiome, including in the nasal passageways (Hardy et al “Corynebacterium pseudodiphtheriticum Exploits Staphylococcus aureus Virulence Components in a Novel Polymicrobial Defense Strategy” mBio, 2019 January-February; 10(1): e02491-18). The radiant exposure level of UVC to destroy most of the common bacteria found in human body cavities was studied by Coohill and Sagripanti (“Overview of the Inactivation by 254 Nm Ultraviolet Radiation of Bacteria With Particular Relevance to Biodefense” Photochem Photobiol September-October 2008; 84(5):1084-90). This research showed that for a 1-log kill, almost all tested microorganisms needed 30-50 J/m2, or 3-5 mJ/cm2. Some required less, some more. For a 4-log kill, most required 100-140 J/m2 or 10-14 mJ/cm2. Based on these studies, coupled with the recent reports from Buonanno and others at Columbia, there appears to be a window of UVC treatment dosage between about 1 mJ/cm2 and 3 mJ/cm2 where pathogens such as coronaviruses can be inactivated without destroying the bacteria making up the normal microbiome in the nasal and respiratory passageways.

There is therefore a need for a method and apparatus to enable an individual or clinician to safely administer doses of UV light having wavelengths between about 200 nm and 230 nm to a body cavity, skin surface, skin lesion or wound. There is a further need for a method and apparatus to inactivate pathogens such as viruses in body cavities without destroying the normal microbiome.

### SUMMARY

In a preferred embodiment, the present invention comprises an energy source, an ultraviolet light emitter, an insertion portion, a user interface and a control circuit. The energy source is preferably a battery, either single-use or rechargeable. The UV light emitter is preferably a UV LED or flat excimer lamp. The insertion portion is of a size and shape to fit within the intended body cavity and configured to direct the UV light to the target tissue. The user interface is a simple on/off button or switch. For the application to the nasal passages, the insertion portion may be a single rod-like element for insertion into one nostril at a time, or it may consist of a bifurcated rod shape for insertion into both nostrils simultaneously. For the application to other body cavities such as the ears, mouth or vagina, appropriately shaped insertion portions may be fashioned by those skilled in the art.

Certain embodiments preferably include certain safety features. One safety feature is a partitioning element which creates a barrier around the region being treated, such that UV light is substantially prevented from leaking from the region, thus reducing exposure of non-target tissue, including the eyes, to UV light. Another safety feature is a visible light sensor located adjacent to the insertion portion, configured to discriminate between light and dark environments. If the user attempts to turn on the UV light while the light sensor senses the presence of ambient visible light, the control circuit will not enable the UV LED. Another safety feature is a timer, configured to count the time that the UV LED is enabled. The control circuit may be configured to disable the UV LED after a certain period of time, such time being set to provide effective disinfection or sterilization while staying below a certain exposure time beyond which may be dangerous for the target tissues. The timer may not only count the time for each dose administered to the target tissue, but may also count the cumulative dosage. The control circuit may limit the cumulative dosage on a daily, weekly, monthly or yearly basis based on safety guidelines or specific needs of a patient.

In yet another variation of the preferred embodiment, the UV emitter is one or more UVC LEDs emitting wavelengths between about 200 nm and 235 nm. Since such LEDs are highly inefficient, certain embodiments include thermal management features to keep operating temperatures within safe limits.

In another preferred embodiment, the UV emitter is an excimer lamp. In one specific embodiment, the lamp is a KrCl excimer emitting peak wavelengths centered around 222 nm. Since KrCl excimer lamps also emit wavelengths that fall outside of the desired range (e.g., 200 nm-235 nm), filters may be used to block unwanted shorter and longer wavelengths.

In a related embodiment, the UV emitter is a KrBr excimer lamp emitting wavelengths centered around 207 nm. Filters may be used as mentioned previously to block unwanted wavelengths from reaching the target tissue.

In another set of embodiments, the UV emitter may be a xenon or deuterium lamp, both of which emit light energy across the UV spectrum as well as into the visible range. Filter elements may be configured to block unwanted wavelengths, such as those outside the desired portion of the UV spectrum. A portion of visible light may be allowed to pass through the filter elements, for example to provide users visual confirmation that the treatment is being applied. Such visible light may also serve as a safety feature, for example, to stimulate a natural eversion response from the user or those nearby, in the event the device is used in such a way that some or all of the light energy is emitted outside the target cavity. In a specific embodiment, the UV emitter is a miniature xenon flashlamp module.

In several preferred embodiments, the device is inserted into a body cavity and delivers a dose of UVC between about 0.1 mJ/cm2 and 10 mJ/cm2 and preferably between about 1 mJ/cm2 and 3 mJ/cm2 in order to inactivate target viruses such as SARS-CoV-2 and other coronaviruses without killing a significant portion of the bacteria making up the normal microbiome.

In yet another embodiment, the device is configured to enter the auditory canal and to deliver UV to the outer and middle ear to destroy bacteria causing ear infections.

In another embodiment, the device is designed with a small-profile insertion portion that can slide into tooth pockets and first measure the depth of the pocket and then apply UV therapy to diseased pockets.

In a further embodiment, the device is configured to disinfect the skin, skin lesions, wounds, canker sores and surgical fields.

### BRIEF DESCRIPTION OF THE DRAWINGS

FIG. 1 is a combined schematic side view and block diagram of an exemplary embodiment of the invention;

FIG. 1A is a front view and block diagram of the embodiment of FIG. 1;

FIG. 2 is a combined schematic side view and block diagram of an alternative embodiment of the invention;

FIG. 3 is a schematic front view of a light source in an embodiment of the invention;

FIG. 3A is a side view of the light source of FIG. 3;

FIG. 4 is a combined schematic top view and block diagram of an alternative embodiment of the invention;

FIG. 5 is a schematic view of the insertion portion of an embodiment of the invention;

FIG. 6 is a combined schematic top view and block diagram of an alternative to the embodiment of FIG. 4;

FIG. 7 is an electrical schematic of an exemplary power supply;

FIG. 8 is a block diagram of the controller portion of an embodiment of the invention;

FIG. 9 is a combined schematic top view and block diagram of an exemplary embodiment of the invention;

FIG. 9A is a front view and block diagram of the embodiment of FIG. 9;

FIG. 10 is a combined schematic top view and block diagram of an exemplary embodiment of the invention;

FIG. 11 is a combined schematic top view and block diagram of an exemplary-embodiment of the invention;

FIG. 12 is a block diagram of a frequency-doubling UV light source;

FIG. 12A is a schematic side view of a laser diode;

FIG. 13 is a combined schematic top view and block diagram of an embodiment which incorporates the light source of FIG. 12;

FIG. 14 is a combined schematic top view and block diagram of an embodiment which incorporates an alternative light source;

FIG. 15 is a schematic top view of an embodiment including a partition element;

FIG. 15A is a schematic front view of the embodiment of FIG. 15;

FIG. 16 is a schematic top view of an embodiment including an alternative partition element;

FIG. 17 is a schematic top view showing the partition element of FIG. 15 further comprising a spring element;

FIG. 18 is a schematic top view showing the incorporation of a cap;

FIG. 18A is a schematic front view of the embodiment of FIG. 18;

FIG. 19 is a schematic top view of an embodiment to treat multiple cavities;

FIG. 20 is a schematic top view of an alternative embodiment to treat multiple cavities;

FIG. 21 is a schematic top view of the embodiment of FIG. 20 further incorporating partition elements;

FIG. 22 is a schematic top view of the embodiment of FIG. 20 further incorporating magnets;

FIG. 23 is a schematic top view of an embodiment of the device shown inserted into a nostril in a partial cutaway view of a nose;

FIG. 24 is a schematic top view of an embodiment of the device shown inserted into both nostrils in a partial cutaway view of a nose, further incorporating a retaining clip;

FIG. 25 is a schematic side view of an embodiment of the invention shown inserted into a nostril in a section view of the nose and mouth of a patient;

FIG. 26 is a schematic side view of an embodiment of the invention shown inserted into the mouth in a section view of the nose and mouth of a patient;

FIG. 27 is a schematic side view of an embodiment of the invention shown inserted into the auditory canal of a section view of the ear of a patient;

FIG. 28 is a schematic side view of an embodiment of the invention shown inserted into a tooth pocket in a section view of a tooth and gum region of a patient;

FIG. 28A is a front view of the embodiment of FIG. 28;

FIG. 29 is a schematic view of an embodiment for treating skin lesions; and

FIG. 30 is a schematic view of an embodiment for treating wounds.

### DETAILED DESCRIPTION

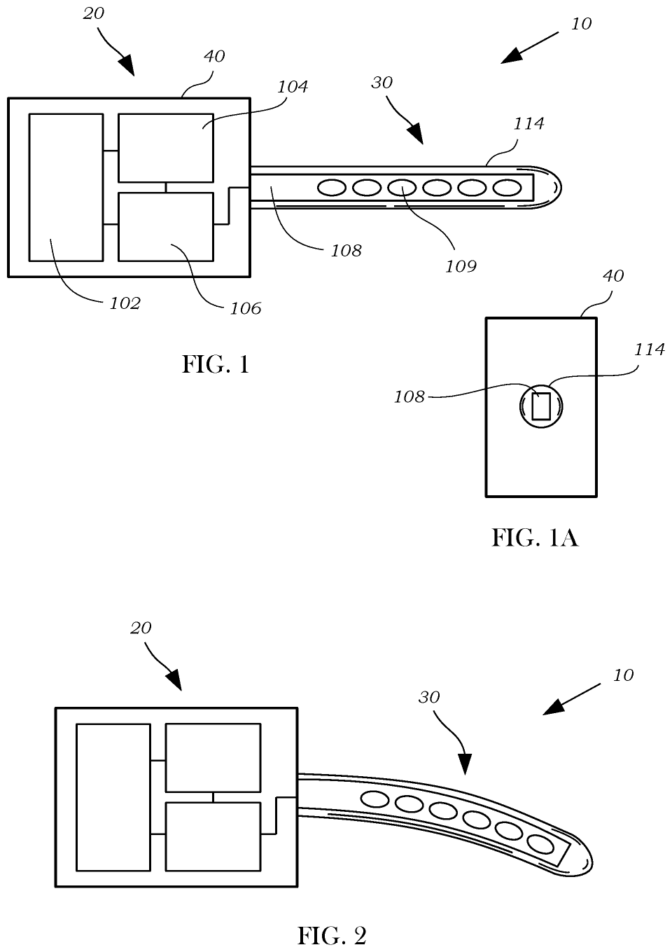

FIG. 1 shows a top view of a preferred embodiment of the device 10, comprised of a main module 20 and an insertion portion 30. FIG. 1A is a front view of device 10, showing a microplasma light source 108 centered axially with respect to a transparent cover 114. Main module 20 houses a battery 102, a power converter 104 and a controller 106. Microplasma light source 108 is fabricated with an array of excimer microcavities 109 that emit ultraviolet (UV) light. Light source 108 can be tuned to emit UV in various wavelengths. In the preferred embodiments, the microcavities use KrBr to emit UV centered around 207 nm, or KrCl to emit UV centered around 222 nm.

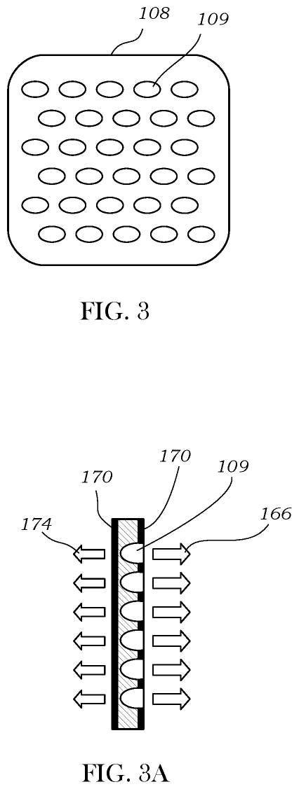

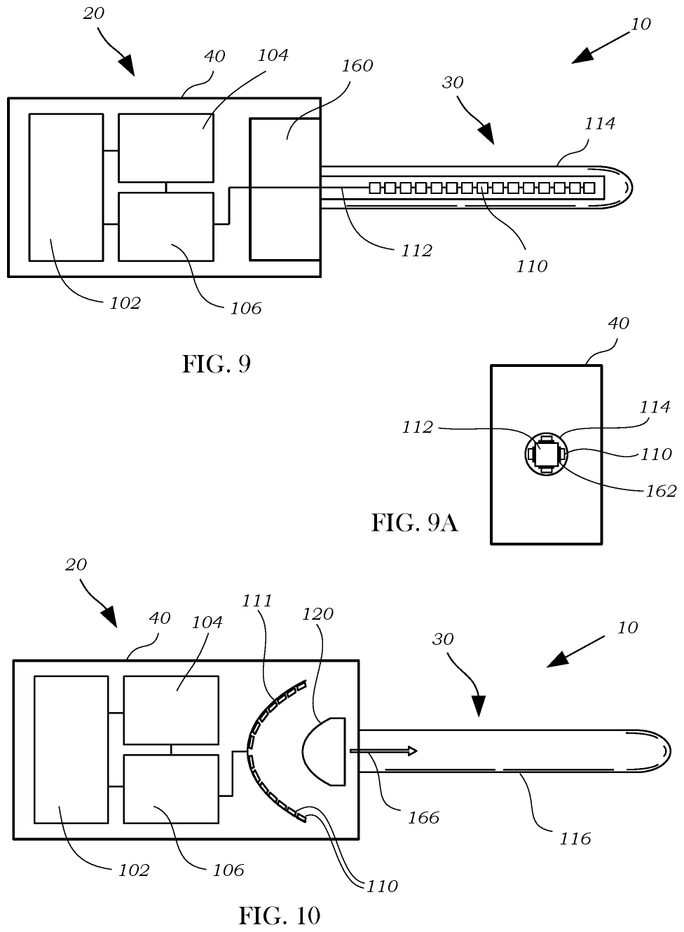

Microplasma light source 108 may be a barrier dielectric discharge source such as those fabricated by Eden Park Illumination (Champaign, Ill. 61821). Eden Park flat excimer lamps emit light energy primarily from the front face but also from the back face. The optical power output from the back face is typically about 70% of that emitted from the front face. (Refer to FIGS. 9 and 9A.) Presently, the Eden Park flat excimer lamps in the 222 nm category emit about 4 mW/cm2, based on the 25 cm2 standard products that they currently offer. For the embodiment depicted in FIGS. 1 and 2, a linear array of microplasma cavities would be required, which can be achieved by adjusting the layout of the microcavities and the electrode array.

As discussed previously, the preferred radiant exposure level is between about 0.1 mJ/cm2 and 10 mJ/cm2, with 1-2 mJ/cm2 being the most preferred. For the convenience of the user, most applications of the UV light to the body cavity are preferably of a duration of ten seconds or less. With an Eden Park flat excimer lamp emitting about 4 mW/cm2, the treatment objective of 1-2 mJ/cm2 could be achieved in less than one second of treatment. A fast treatment such as this would allow a user to quickly apply an effective treatment from the device to their nasal passage or oral cavity without discomfort.

The thickness of microplasma light source is about 6 mm or less (referring to the horizontal dimension shown in FIG. 1A). The width of the array (vertical dimension as shown in FIG. 1A) may be slightly more than 6 mm, such that when surrounded by transparent cover 114, the overall outer diameter is less than 10 mm (which is roughly the minimum diameter of a typical nostril). Transparent cover 114 is made from a material that passes the desired wavelength. For example, it may be fabricated from fused silica (such as Suprasil® made by Heraeus Conamic, 100 Heraeus Blvd. Buford, Ga. 30518), calcium fluoride, magnesium fluoride, lithium fluoride and sapphire. Fused silica is used in a preferred embodiment because it is more than 80% transparent to wavelengths longer than 200 nm, and is relatively inexpensive. Highly purified calcium fluoride (CaF2) may also be used. It has a very low UV absorption, high homogeneity, low birefringence, relatively high hardness (compared with other fluoride materials), high physical stability, and high optical damage threshold. However, it is brittle, naturally anisotropic, hygroscopic and more expensive than fused silica. Magnesium fluoride (MgF2) and lithium fluoride (LiF) also have good UV transparency, but share some of the other disadvantages of calcium fluoride. Artificial diamond is less transparent below about 230 nm and is very expensive. TOPAS® cyclic olefin copolymer (Topas Advanced Polymers, Raunheim, Germany) is transparent down to 222 nm and has the advantage of being moldable but does degrade after repeated exposure to UV.

FIG. 2 is a side view of an embodiment similar to that of FIG. 1, except the insertion portion 30 is curved to match the anatomy of a target body cavity. Referring to FIGS. 25 and 26, it will be appreciated that the embodiment of FIG. 2 using curved insertion portion 30 may be better adapted to enter the nasal and/or oral cavities and align with the natural anatomical shapes.

The microplasma array may also be situated outside of the insertion portion. FIG. 3 shows one embodiment of a microplasma light source 108 in the form of a flat excimer lamp including an array of microcavities 109. By way of example, light source 108 may be about 25 cm square. FIG. 3A is a side view of the light source 108 of FIG. 3, showing the microcavities 109 and electrodes 170. Microcavities 109 contain a gas such as KrBr or KrCl which, when excited, emit UV at wavelengths 207 nm and 222 nm, respectively. Most of the UV light is emitted from the front side, as indicated by light ray 166. However, some additional UV light may be emitted out the back side, as indicated by light ray 174.

FIG. 4 shows an embodiment with a flat excimer light source 108 situated in main module 20. A reflector 164 is positioned behind light source 108 to reflect light rays 174 coming out of the back of light source 164. An optional optical coupler 120 collects light energy from light source 108 and channels it into an entry point of insertion portion 30. In embodiments such as this, insertion portion 30 essentially becomes a light guide 116, channeling and dispersing the light energy to the targeted surfaces within the body cavity. Preferred materials for light guide 116 include the same materials described previously for the transparent cover 114. Alternatively, light guide 116 may be a hollow tube with a tapered tip. Such a hollow tube made be made from a transparent material, in which case either the internal or external surfaces may be plated with a reflective coating. The coating may be applied with partial breaks or voids where light rays can escape. As such, the coating pattern can be fine-tuned to enable dispersion of the light in desired directions and locations. The hollow tube may also be made from metal such as stainless steel, with the inner surface highly polished and/or plated with a reflective coating. A pattern of holes in the metal tube can allow light to escape in desired locations. It will be appreciated that a combination of a hollow metal tube with a transparent core or cap material is possible.

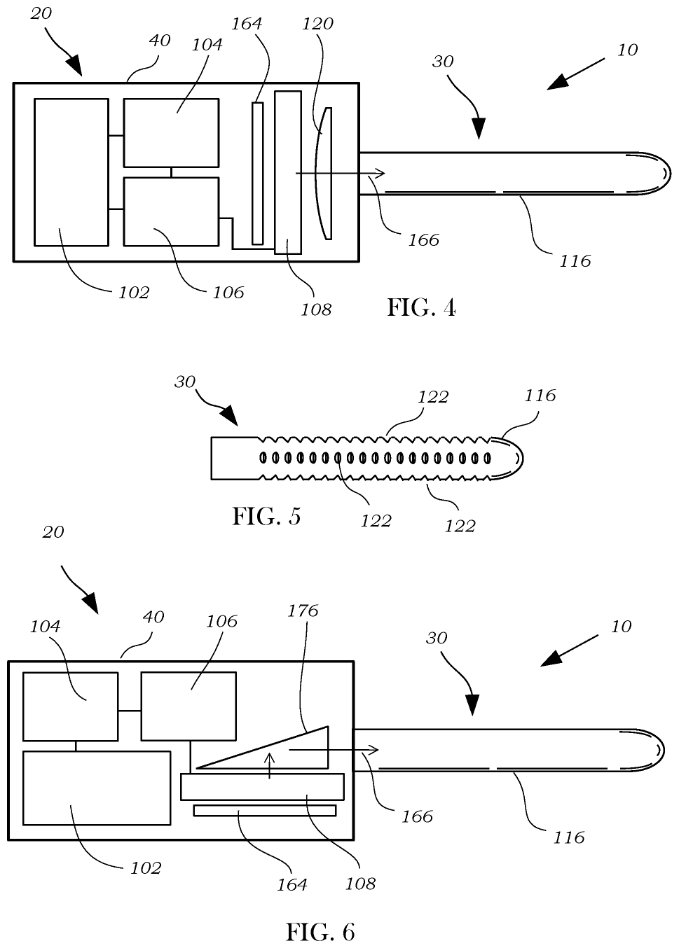

Referring now to FIG. 5, insertion portion 30 is shown as a light guide 116. Depending on the level of collimation of light rays 166 entering the proximal end of light guide 116, light rays 166 will scatter and exit the outer surface of light guide 116 in various ways. In order to better direct the distribution of light across the targeted surfaces in the body cavity, light guide 116 may have an array of diffusing elements 122 along its length. Such elements redirect light rays 166 traveling primarily axially along the length of light guide 116 to a more oblique angle or radially away from the surface of light guide 116. Diffusing elements 122 may be molded or machined cuts in the surface of light guide 116, with or without a reflective material. Alternative diffusing or dispersing methods may include reflective particulates, bubbles, and the like embedded in light guide 116.

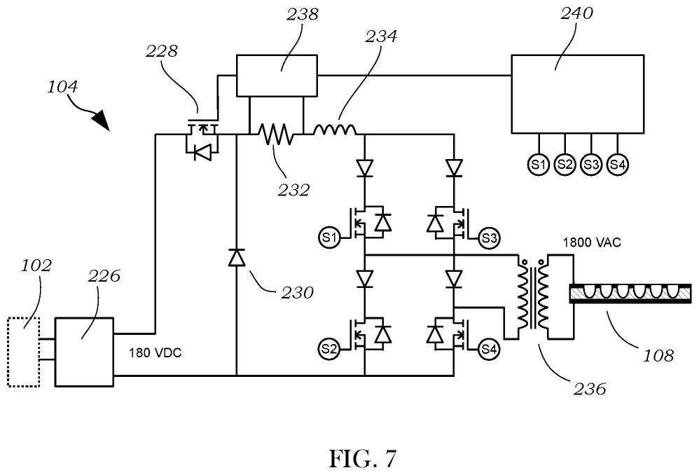

FIG. 6 shows an alternative construction to the embodiment shown in FIG. 4, with microplasma array 108 and reflector 164 turned orthogonally relative to that of FIG. 4. This allows a slimmer cross-sectional profile of main module 20, thereby allowing the device 10 to be easier to carry in a pocket or purse or for storage. A right angle coupler 176 may be one or more optical elements such as a reflecting prism made of fused silica (or other appropriate UV-transparent material discussed previously) to reflect the light rays 166 into the entry point of insertion portion 30. The reflecting material on the prism must be able to reflect the deep UV light, so materials such as enhanced aluminum (Edmund Optics, Barrington, N.J.), high-density aluminum (Duval Mirrors, CVI Lasers, Albuquerque, N. Mex.), low-loss dielectric (Newport Corp, Irvine, Calif.), and the like must be employed.

Light source 108 must be driven by a high-voltage power supply. A preferred embodiment of power converter 104 appropriate to drive microplasma array 108 is shown in in FIG. 7. When a battery 102 is connected to DC-to-DC converter 226, the battery voltage is first converted to a higher voltage. In one preferred embodiment, the battery voltage is in the range of 3.0-3.6V, and the output of the DC-to-DC converter is about 180V as indicated in FIG. 7. MOSFET 228 passes the high voltage through sense resistor 232 and buck inductor 234 to the full bridge circuit made up of four more MOSFETs. Hysteresis circuit 238 sense the voltage across sense resistor 232 and modulates the on-resistance of MOSFET 228. Pulse width modulation circuit 240 controls the on/off pattern of the four MOSFETs making up the bridge via signals S1 through S4 as indicated, thereby alternating the direction of current flowing through the primary winding of step-up transformer 236. The secondary winding of transformer 236 is connected to the electrodes of microplasma array 108. In one preferred embodiment, the peak output voltage on the secondary of transformer is between about 1.2 kV and 2.0 kV, and is preferably around 1.8 kV as indicated in FIG. 7. The frequency of switching signals S1-S4 may vary from about 15 Hz to 100 Hz but in a preferred embodiment may be between 20 and 30 Hz.

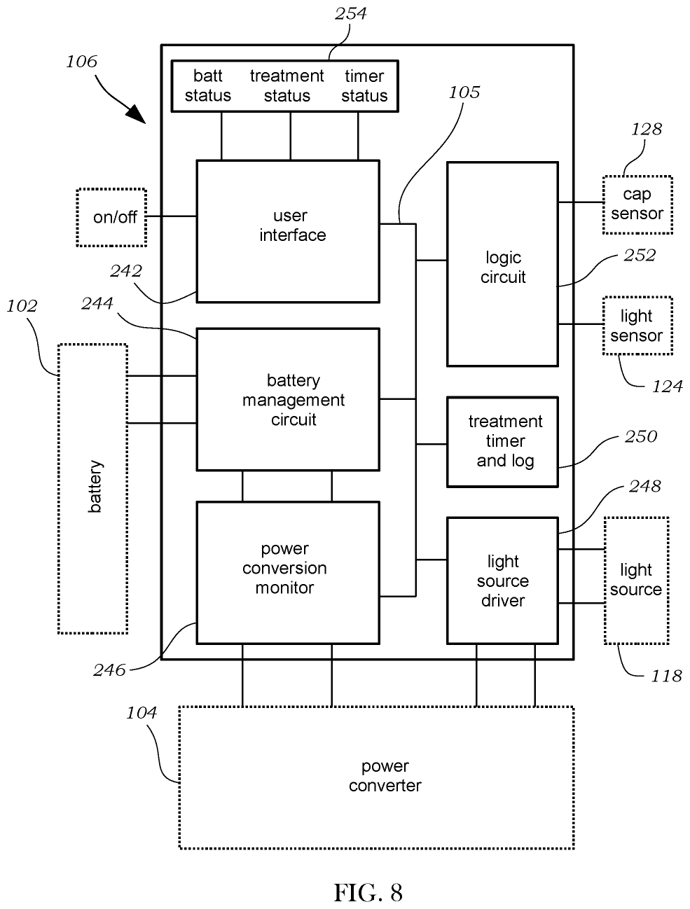

Operation of device 10 is managed by controller 106, shown in block diagram form in FIG. 8. User interface 242 is preferably simple. In the embodiment depicted in FIG. 8, there is an on/off switch to turn device 10 on or off. Status indicators 254 may include LEDs to provide users with the current status of the battery, treatment and timer(s). Battery 102 is connected to battery management circuit 244, which monitors battery voltage and remaining capacity, controls the charging process, and regulates the voltage provided to other parts of controller 106. Circuit 244 may be a single-chip or multichip solution known to those skilled in the art from vendors such as Texas Instruments (Dallas, Tex.) and Maxim Semi (San Jose, Calif.). Power conversion monitor 246 oversees the performance of power converter 104, and may have the ability to shut down converter 104 if certain thresholds of current, voltage, temperature or the like are exceeded. Power converter 104 may be of the type described in FIG. 7 for microplasma light sources, or it may be a switched mode power supply or the like when driving a light source that does not require high voltage, such as any of the LED embodiments. Treatment timer and log 250 keeps track of the amount of time the light source has been enabled, and may further keep a log of the date, time and duration of each treatment. Pre-determined limits of treatment time and daily, weekly and monthly usage may be programmed into timer 250 and/or logic circuit 252 in order to ensure that users do not exceed certain safety limits. Logic circuit 252 receives inputs from user interface 242, battery management circuit 244, power conversion monitor 246 and treatment timer 250 via data bus 105, and sends signals to light source driver 248 via bus 105 to either turn on or off a light source 118.

FIG. 9 shows an embodiment similar to that of FIGS. 1 and 2, except that light source 118 is an array of LEDs 110 attached to a stem 112. As shown in FIG. 9A, stem 112 is square in cross-section with LEDs 110 attached on each side. It will be appreciated that other cross-sections are possible, such as triangular, rectangular and the like. LEDs 110 preferably emit in the UV wavelength range of 200 to 400 nm. LEDs are readily available in wavelengths from 250 nm to 400 nm and beyond. In a preferred embodiment, LEDs 110 have peak emission between 200 nm and 230 nm. The deep-ultraviolet (DUV) LED emitting 222 nm produced by Riken (Sendai, Miyagi, Japan) is based on AlGaN technology and has been demonstrated to emit up to 14-20 uW at approximately 0.01-0.02% efficiency using about 20 mA drive current. Efficiency of DUV LEDs can be increased by adding a transparent p-AlGaN contact layer, but this increases contact resistance, resulting in a significant increase in operating voltage. Riken has been able to incorporate a photonic crystal (PhC) reflector on a p-GaN contact layer to achieve low contact resistance and high-reflectivity on the p-contact layer. Using techniques such as this, it is believed that over the next decade, great strides will be made to improve output level and efficiency.

The Riken 222 nm DUV LEDs will be used as an example for enablement of the present invention. For the application involving the nasal cavity, assuming a depth of insertion of about 25 mm and a nostril diameter of about 10 mm, the circumferential area of treatment is estimated as π*d*h, where d is the diameter of 10 mm and h is the depth of about 25 mm, or about 7.8 cm2. If the LEDs emit 20 uW, and 2 mJ/cm2, or 0.2 mW/cm2 are needed, assuming a ten-second treatment, then the total power output across 7.8 cm2 is 1.6 mW. If each LED emits 20 uW, then 80 LEDs are needed. In the configuration shown in FIG. 4, that suggests about 20 LEDs are needed on each of the four sides (assuming very little loss through cover 114). Putting that many LEDs on a single stem 112 may generate heat, so stem 112 may be made from a material with high thermal conductivity such as copper, aluminum or the like. Further, the LEDs 110 may be bonded to stem 112 with a thermally conductive material such as thermal epoxy, preferably a material such as Arctic Silver Premium Silver Thermal Epoxy (Arctic Silver, Visalia, Calif.). Simulations show that even with these materials, the LEDs and stem will get hot after a few seconds. To counteract this, a large mass can be coupled to stem 112.

In FIG. 9, a heat sink 160 is shown to address this heat removal need. Heat sink 160 may be made from a material such as copper or aluminum and may have cooling fins or may have an integrated thermoelectric cooler to reduce its initial temperature and to manage the heat load during treatment. A CAD simulation using 60 LEDs (15 on each side), with a 25 mm stem having a square cross-section of 5 mm on each side, and a cubic heat sink of approximately 0.6 cm on each side kept at an initial temperature of around 0° C., shows that the distal tip of the stem can reach over 60° C. after ten seconds, and the distal-most LED can reach over 90° C. Assuming the cover 114 provides significant thermal insulation, this design would likely be acceptable for single treatments (assuming the LEDs can handle 90° C. without losing efficiency). However, if multiple treatments over a short period of time are desired, or if the full 80 LEDs are arrayed onto the stem, then additional measures may be taken to cool the stem and the LEDs. Such measures may include adding an airflow path to circulate air through the inside of cover 114 or providing internal cooling channels within stem 112 to circulate cooling fluid, for example. Further methods for improving efficiency of LEDs 110 may include utilizing flip-chip morphology and filling the internal cavity with transparent sol-gel fluoro siloxane hybrid material as described by Bae et al (RSC Adv., 2016, 6, 26826) or silicone oil such as KF-96-50CS (Shin-Etsu Chemical Co., Ltd, Tokyo, Japan). This silicone oil not only enhances the optical output of the LEDs but also improves heat dissipation and therefore improves efficiency and reduces thermal degradation. Yet another method of improving the efficiency of the deep UV LEDs 110 is to apply external bending by integrating them onto a pre-defined curved packaging substrate, as described by Shervin et al (J Phys D: Appl Phys 51, 2018; 105105). For example, stem 112 could be slightly curved, which may prove beneficial (as described for the microplasma embodiment depicted in FIG. 2).

An alternative to putting LEDs 110 along a stem within insertion portion 30 is to position them within the main module 20 proximal to the insertion portion (similar to FIG. 4) with optical coupler 120 gathering the light from the LEDs 110 and channeling their radiant output 166 to the base of the insertion portion 30, in which case insertion portion 30 essentially becomes a light guide 116 to deliver the light energy to the desired body cavity surfaces. Another alternative to channel the light output from LEDs 110 to the light guide 116 may include using a reflecting prism made of fused silica (or other appropriate UV-transparent material discussed previously) to redirect light from LEDs 110 to the entry point of the light guide 116 (similar to the arrangement shown in FIG. 6). The reflecting material on the prism must be able to reflect the deep UV light, so materials such as enhanced aluminum (Edmund Optics, Barrington, N.J.), high-density aluminum (Duval Mirrors, CVI Lasers, Albuquerque, N. Mex.), low-loss dielectric (Newport Corp, Irvine, Calif.), and the like must be employed. The LEDs 110 may be arrayed in a nonlinear pattern to maximize the amount of light entering light guide 116; such patterns being angled, curved, parabolic or the like.

FIG. 10 shows an embodiment where LEDs 110 are situated in main module 20 and arranged on a parabolic substrate 111, with optical coupler 120 gathering the light from the LEDs and channeling it into the base of light guide 116 as shown by light ray 166. As described previously, the curved substrate 111 on which LEDs 110 are arrayed may actually improve efficiency of the LEDs.

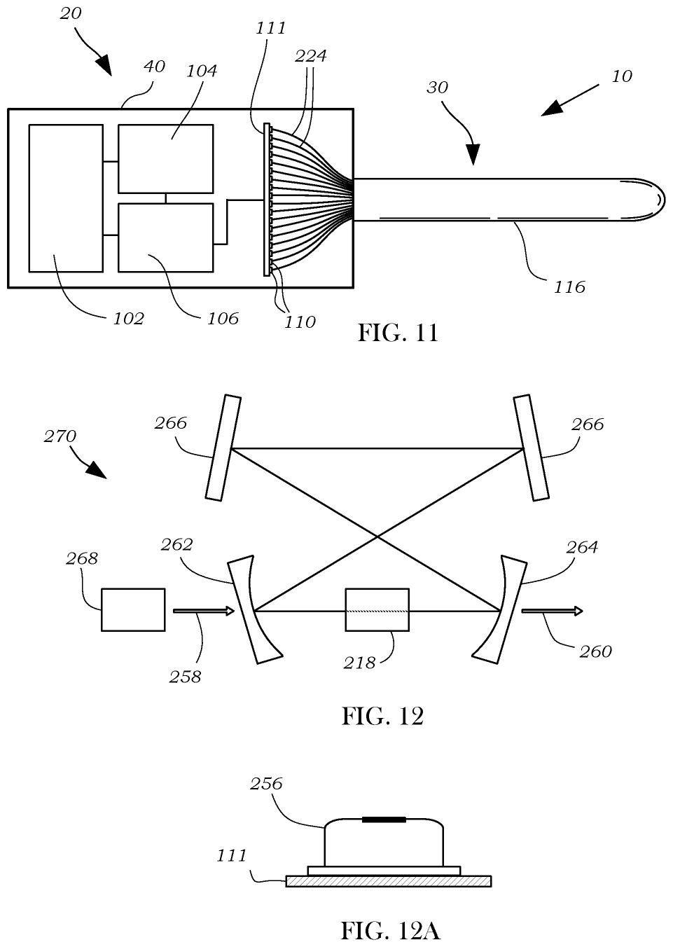

FIG. 11 shows another method of redirecting the light from LEDs 110 to light guide 116 using fiberoptics. Silica fiber bundles may be used, preferably with hydrogen-infused cores to resist degradation due to solarization. Using fiberoptics, the LEDs can be arranged in any pattern, and the fiber bundle can be tailored to channel the LED light output to the entry point of the insertion portion with minimal loss. Also, liquid filled light guides can transmit deep UV down to 222 nm (Oriel®, Newport, Irvine, Calif.). FIG. 11 shows an array of LEDs 110 arranged on a flat substrate 111 and fiberoptic array 224 configured so that the light output from the LEDs is channeled to the proximal end of light guide 116. It will be appreciated that fiberoptic array 224 may continue into, or otherwise be integrated into, light guide 116. Light guide 116 may in fact be fiberoptic bundle 224, encapsulated by an appropriate material such as silicone, so long as the distal ends of each of the fibers is brought to the surface of light guide 116, since silicone is not transparent to deep UV. By doing so, the UV light may be dispersed uniformly along and around light guide 116. Alternatively, fibers from bundle 224 may be embedded in a rigid DUV transparent material such as fused silica.

Battery 102 may be any type having an appropriate capacity and current output capability. The DUV LEDs produced in the Riken lab require a drive current of 20 mA per LED and a forward voltage of between 6V and 12V. For an embodiment with 60 LEDs, 1.2A of LED drive current is needed. If a rechargeable lithium-ion type battery is chosen, the nominal voltage ranges from about 2.5V to 3.6V. Assuming a useful minimum voltage of 3.0V and an LED forward voltage of 12V, then a DC-to-DC converter is needed with a 12:3 ratio, or 4X. Assuming an 80% conversion efficiency, the 1.2 A delivered to the LEDs will require about 4.8 A out of the battery. Few small-format rechargeable batteries can deliver that current load. One such battery is model LPHD452535 from LiPol Battery Co Ltd (Shenzen China). This battery is 35 mm×25 mm×4.5 mm thick and has a 250 mAhr capacity with a discharge current capability of 20C, or 5 A. This battery could fit nicely in a pocket-sized, handheld device. As far as the number of treatments that the battery could deliver on a single charge, that can be estimated by dividing 250 mA-hr by the current draw of 4.8 A, which comes to about 187 seconds. At ten seconds per treatment, that would allow about 18 treatments (or slightly less as a result of additional overhead current needed to run other parts of the controller).

The battery requirements for the embodiments of FIGS. 1-7 which use the microplasma excimer light source can be estimated by noting that the 25 cm2 flat excimer lamp emitting 222 nm light offered by Eden Park comes with a 10 W power supply. Assuming the LPHD452535 lithium-ion polymer battery minimum working voltage is 3.0V, to deliver 10 W would require about 3.3 A, which is within the battery's capability. As for the number of treatments the battery can deliver, 250 mA-hr divided by 3 A comes to 300 seconds, or about 30 ten-second treatments.

Yet another method of generating deep UV in the wavelengths of interest is to use a blue or violet LED emitting light in the wavelength range 400-460 nm that is passed through a non-linear optical system, where the light is up-converted to its second harmonic, which reduces the wavelength to half, or 200-230 nm. In a preferred embodiment, the non-linear optical system has high-efficiency second-harmonic generation using a crystal of Barium Borate (BaB2O4), abbreviated BBO. By way of example, the crystal may be a β-BBO crystal supplied by CryLink (Shanghai, China), which shows a phase-matching angle θ of 90° for 400 nm excitation and 200 nm emission, and θ of 79° for 415 nm excitation and 207 nm emission.

Ultraviolet energy may also be generated using lasers, as described by RP Photonics online encyclopedia (www.rp-photonics.com). Free electron lasers can emit ultraviolet light of essentially any wavelength, and with high average powers. However, they are very expensive and bulky sources, and are therefore not practical for a portable device or for consumer applications. Apart from such direct-wavelength ultraviolet lasers, there are ultraviolet laser sources based on a laser with a longer wavelength (in the visible or near-infrared spectral region) and one or several nonlinear crystals for nonlinear frequency conversion. For example, the wavelength of 355 nm can be generated by frequency tripling the output of a 1064-nm Nd:YAG or Nd:YVO4 laser. Similarly, 266-nm light is obtained with two subsequent frequency doublers, which in effect quadruple the laser frequency.

Diode lasers can also be equipped with nonlinear frequency conversion stages to produce UV light. For example, one may use a continuous-wave, near-infrared laser and apply resonant frequency doubling twice, arriving at wavelengths around 300 nm. A main attraction of this approach is that a wide range of wavelengths is accessible, with no limitations to certain laser lines. Frequency quadrupling is a process of nonlinear frequency conversion where the resulting optical frequency is four times that of the input laser beam, which means that the wavelength is reduced by a factor of 4. This can be accomplished with two sequential frequency doublers. Another possibility is to use a single frequency doubler and two sum frequency generation stages for mixing with residual pump light. A commonly used frequency quadrupling configuration begins with a continuous-wave or pulsed Nd:YAG laser at 1064 nm for generating 532-nm light in a first frequency doubler stage (based e.g. on LBO=lithium triborate) and then 266 nm in a second stage (based e.g. on CLBO=cesium lithium borate).

Toptica Photonics AG (Munich, Germany) produces the SHG Pro system which provides frequency-quadrupled continuous-wave diode and fiber lasers based on two cascaded frequency-doubling stages. Available wavelengths range from 190 nm to 390 nm with up to 10 nm of tuning. The system offers long-term stable operation and higher powers at DUV wavelengths. However, it is expensive and bulky and not practical for a portable device.

Photon Systems (Covina, Calif.) produces a DUV laser at 224.3 nm in a compact, relatively inexpensive system (as compared to conventional lasers). The self-contained, integrated, laser controller enables remote computer control for ease of operation and flexible data collection via LabView software. With an input power less than 10 W the need for water cooling and other thermal management issues is eliminated. The laser reaches full power in less than 20 microseconds from a cold start with output levels to 100 mW.

Another approach to generating deep UV is to use a discrete laser diode as the primary light source, and to use frequency doubling to generate the UV wavelength of interest. FIG. 12 shows a typical frequency-doubling light source 270. Pump source 268 emits light at twice the desired wavelength. That light, called the pump light 258, passes through high reflector mirror 262 and through non-linear crystal 218 to output coupler mirror 264. Light at the wavelength of the pump light 258 is then reflected up to folding mirrors 266 and back to mirror 262, and so on. As the intensity of pump source light builds passing through non-linear crystal 218, a second harmonic of the pump source frequency begins to develop. Mirror 264 is configured to reflect the pump source frequency and pass the second harmonic frequency. As shown, the frequency-doubled light 260 passes through mirror 264. Note that this is just one example of a frequency-doubling scheme, which happens to use a folded mirror technique. Other methods are known to those skilled in the art. By way of example, pump source 268 may be a 447 nm laser diode such as part number L450G1 from ThorLabs (Newton, N.J.), and crystal 218 may be a β-BBO crystal supplied by CryLink (Shanghai, China). Part L450G1 is packaged in a 9 mm TO package, depicted in FIG. 12A, and is approximately 3 mm in height, so it is small enough to fit inside a compact, handheld device. The optical power output of this laser diode is up to 3 W. The efficiency of conversion through the frequency-doubling process is dependent on many variables. Assuming a low efficiency of 5%, the optical power entering optical coupler is then 150 mW, which is more than enough to meet the desired output levels as described below.

FIG. 13 shows an embodiment incorporating the frequency-doubling light source 270 discussed above. Light source 270 is driven by controller 106 and its output 260 enters light guide 116. Assuming losses in the optical coupling to the light guide and within the light guide of 20% each, the optical power emitted from the light guide toward the surfaces of the targeted body cavity is about 96 mW. To treat an area of 10 cm2, that equates to 9.6 mW/cm2. For a 10-second treatment time, we only need 0.1-0.2 mW/cm2. So there is about a 100:1 margin between available power and required power. Using that to advantage, the laser diode can be pulsed at a duty cycle as low as 1% and still achieve the target optical power output. A low duty cycle will help in managing the heat produced by laser diode 256 as well as increase battery life. It will be appreciated that the efficiency of the frequency-doubling process may be significantly lower than 5%, and the losses in optical coupling and scattering through the light guide may be higher than 20% each. Nonetheless, there is a wide margin to adjust the duty cycle of the laser and still achieve the desired therapeutic dose.

An alternative to using a laser diode as the pump source is to use a blue LED. For example, a 445-450 nm LED such as part number L0F2-B445050002751 from Philips Lumileds may be used. Because light emitted from an LED is not coherent, the efficiency of the frequency doubling scheme is limited, and fine-tuning the phase-matching of the LED and crystal is challenging. The L0F2-B445050002751 LED can emit about 280 mW of optical power. Assuming a 1% efficiency of the frequency doubler, the output to the light guide would be 2.8 mW. As previously assumed, if there is a 20% loss coupling to the light guide and traveling through the light guide, the final light output from the light guide is about 1.8 mW. To treat an area of 10 cm2, that equates to about 0.18 mW/cm2. For a ten second treatment, that results in about 1.8 mJ/cm2, which is 10× the desired dose. That implies that we can either pulse the LED at 10% duty cycle to reduce heat and improve battery life, or we can accommodate a frequency doubling efficiency as low as 0.1%.

Other types of light sources may be used if there is less need for portability, such as for treating skin lesions or nail fungus, for example. Such sources may include arc lamps using various noble gases (argon, neon, krypton, and xenon), for example continuous-output xenon short-arc lamps and long-arc lamps, mercury xenon lamps and hollow cathode lamps (Hamamatsu, Iwata City, Japan). Mercury vapor lamps emit in the deep UV, for example medium pressure UV lamps by Helios Quartz (Novazzano, Switzerland). Deuterium lamps also emit deep UV energy, although they take some time to warm up (e.g., 20 or more seconds). Ushio Inc (Tokyo, Japan) makes an excimer lamp that emits 222 nm, but it is bulky and not intended for handheld or battery-operated use.

Xenon flash lamps may also be used as a suitable source of deep UV for the present invention. For example, Excelitas (Salem, Mass.) produces a compact xenon flash lamp source, part number RSL-2100. It can output up to 2 W of optical power across a broad spectrum from below 200 nm to over 900 nm. For a preferred embodiment of the present invention, a bandpass filter would be utilized to block the unwanted wavelengths below about 200-205 nm and above about 225-230 nm. Such filters require exotic materials and may be fabricated using a metal-dielectric-metal process or a multi-layer Fabry-Perot design. As an example, a 218 nm bandpass filter has been demonstrated using a four-layer Fabry-Perot filter design on a MgF2 substrate (see “Enhanced Aluminum reflecting and solar-blind filter coatings for the far-ultraviolet”, Javier Del Hoyo, Manuel Quijada, NASA Goddard Space Flight Center, full article incorporated herein as reference.) As a first approximation, about 5-10% of the optical power output from a xenon flash lamp is in the 200 nm to 230 nm region. Using the RSL-2100 at maximum output power, that would suggest 0.1-0.2 W of output power in the region of interest.

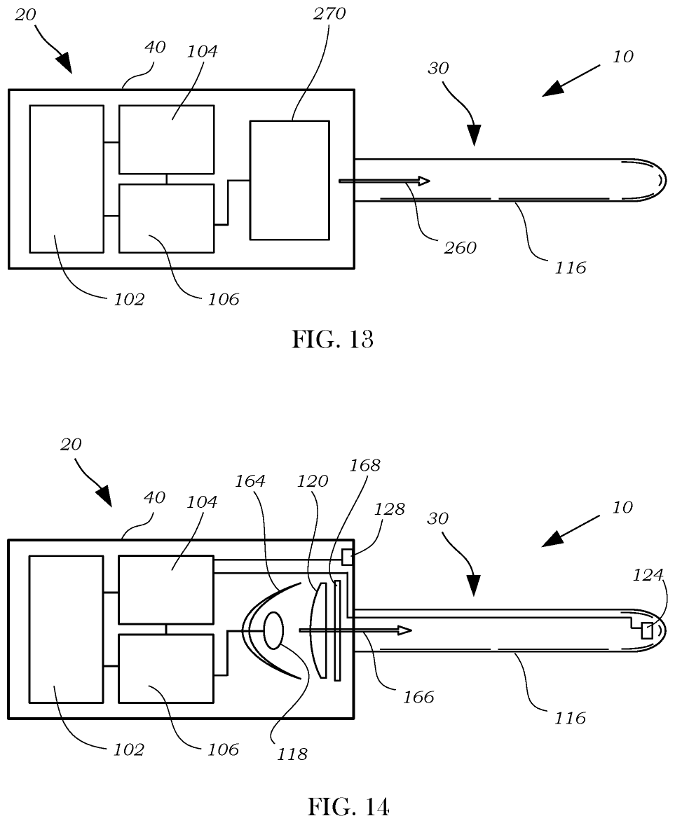

Referring now to FIG. 14, device 10 is shown incorporating light source 118 which may be of any type of light source described herein having a bulb or lamp. A reflector 164 surrounds the back side of light source 118 and directs radiant energy in the direction of the light guide 116. Certain light sources may have an integrated reflector. In either case, reflector 164 reflects UV in the region of interest and may be coated with enhanced aluminum, high-density aluminum, low-loss dielectric or the like. Optical coupler 120 is shown as an optional element to further direct radiant energy from light source 118 toward the entry point of light guide 116. Preferably optical coupler 120 is made from a UV transparent material such as fused silica, MgF2 and the like. Filter 168 is shown, representing the bandpass filter previously described. It will be appreciated that some or all of the filtering may happen on or in optical coupler 120 or on or in the light source 118, or within or on light guide 116.

Using the assumption that light source 118 is the RSL-2100 xenon flash module, reflector 164 is not needed. Optical coupler 120 may be desirable to collect as much optical energy from the output of light source 118 as possible and direct it into light guide 116. Assuming a loss of 20% through coupler 120 and 20% through filter 168 and 20% through light guide 116, the output power in the region of interest drops from 0.1-0.2 W down to 50-100 mW. Assuming the treatment zone is 10 cm2, this implies a range of 5-10 mW/cm2. With a ten second treatment time, that translates to 50-100 mJ/cm2, which is about 50 times the target therapeutic dose discussed earlier. However, this assumes the flash lamp is running at maximum output power, in which case the lamp portion of light source 118 would get very hot. As such, light source can be run at a low duty cycle, such as 10%, reducing the delivered output power to 0.5-1 mW/cm2, so the treatment time could be reduced to just two seconds in order to achieve the target range of 1-2 mJ/cm2.

The embodiment of FIG. 14 shows two additional optional features of device 10. First is a light sensor 124, which is shown at the tip of light guide 116. Sensor 124 may be a visible light sensor, UV sensor or both. A visible light sensor can be used to detect when it is safe to enable the UV light by sensing if there is ambient light leakage into the targeted treatment region (partitioning elements will be discussed in subsequent sections). Sensor 124 may also detect the level of UV traveling through light guide 116 or reflecting from the surrounding walls of the body cavity, as a means of determining the proper functioning of the UV source and light delivery apparatus.

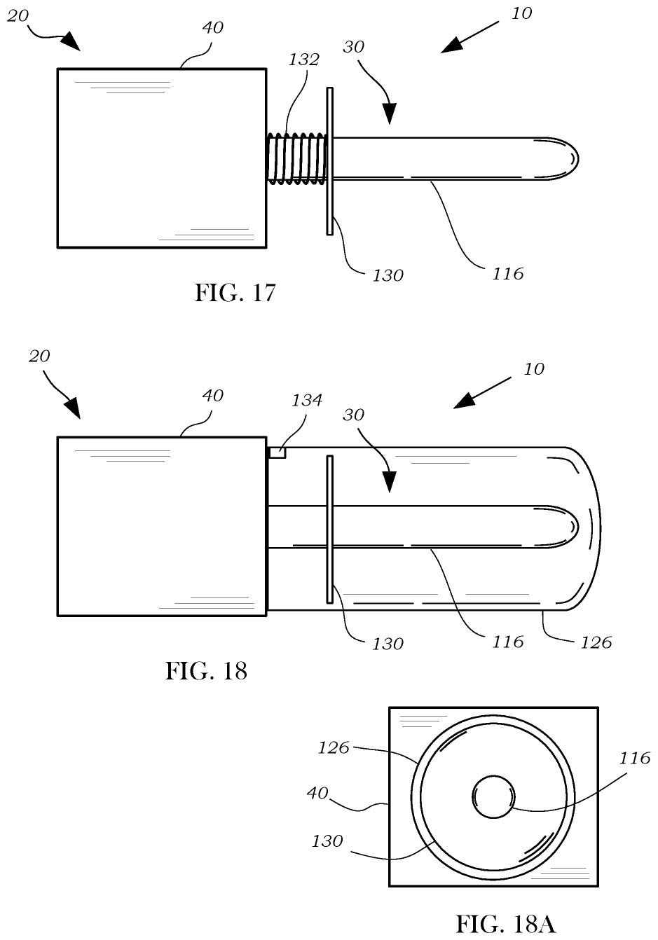

Another optional feature shown in FIG. 14 is a cap sensor 128. The cap is an optional feature depicted in FIG. 18, whose function is to protect insertion portion 30. Cap sensor 128 may be any type of sensor known to those skilled in the art, including a microswitch, optical sensor, proximity sensor, capacitive sensor, reed switch and the like. Signals from cap sensor 128 and light sensor 124 feed into controller 104 as indicated in FIG. 8.

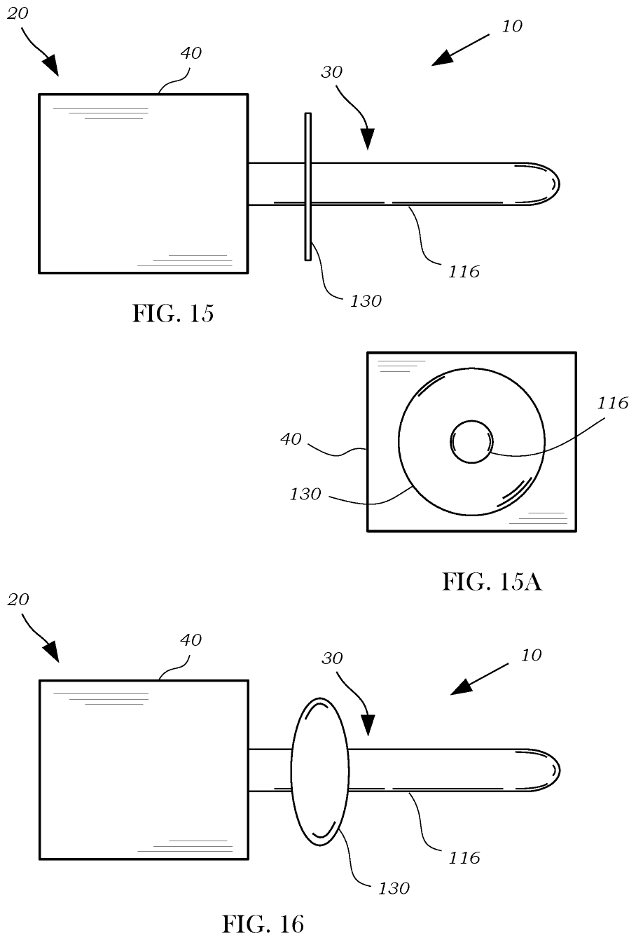

FIG. 15 shows a simplified version of device 10 including a partition 130. Partition 130 serves to block UV light from escaping from a body cavity once insertion portion 30 has been fully inserted into the cavity. Partition 130 may be made from a flexible material such as silicone or polyurethane; however such materials will degrade over time when exposed to deep UV, so partition is preferably easily replaceable. FIG. 15A shows a front view of the device of FIG. 15.

A variation of the device 10 of FIG. 15 is shown in FIG. 16. Partition 130 is of a bulbous form, preferably made of a soft durometer silicone, polyurethane or foam, or may be an inflatable balloon. In such embodiments, partition 130 is intended to give a snug seal around the opening of the body cavity. In the case of treatment to the nasal cavity, partition 130 may partially insert into the opening of the nostril not only to seal and prevent light leakage but also to hold device 10 in place during the treatment period.

FIG. 17 depicts a further refinement of the embodiment of FIG. 15, showing a spring 132 that provides a pushing force to partition 130. In this configuration, partition 130 can tilt and slide along light guide 116 as needed in order to accommodate varying sizes and shapes of noses and nostrils (or other openings to body cavities).

Referring now to FIG. 18, device 10 is shown with a protective cap 126 covering insertion portion 30. Cap 126 is preferably made from a non-UV transparent material such as plastic or metal. FIG. 18A is a front view of device 10 with cap 126 installed. One purpose of the cap 126 is to prevent the insertion portion 30 from being damaged when not in use. A second purpose is to allow device 10 to perform a self-disinfecting step after each use. The method of self-disinfection follows the steps of: first, detecting the attachment of a cap 126 over the insertion portion 30; second, enabling a light source to self-disinfect the insertion portion 30; and, third, initiating a timer which turns off the light source when the timer expires at the end of self-disinfection. Such a self-disinfecting step serves to not only disinfect insertion portion 30 after each use, but also the internal surfaces of cap 126.

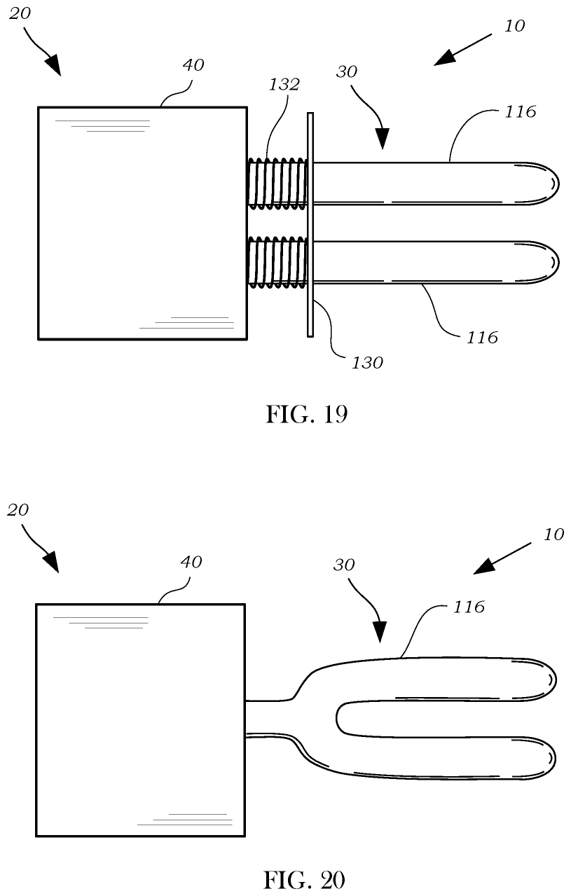

FIG. 19 shows an embodiment of device 10 having two insertion portions 30. This arrangement is particularly suited for treatment of the nose, with one insertion portion going into each nostril. In the embodiment shown, partition element 130 bridges across both insertion portions, and includes a pair of springs 132. Alternatively, each insertion portion 30 could have its own partition, and such partitions could be of the bulbous type shown in FIG. 16, or another other appropriate shape.

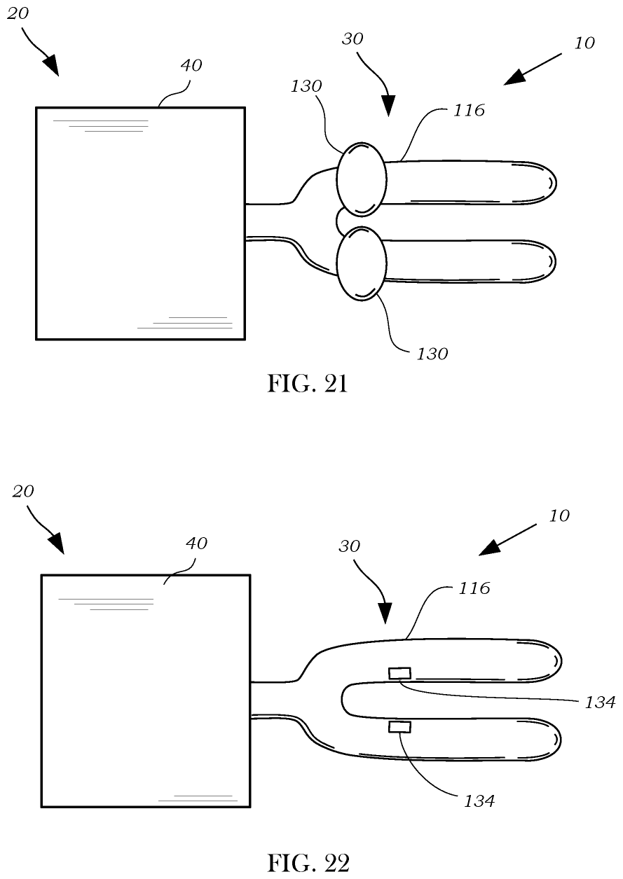

In FIG. 20, insertion portion 30 is shown bifurcating into two branches. The intended use of this embodiment is for treatment of the nasal cavity, with one branch going into each nostril. Partition elements (not shown) can be added in obvious ways to this configuration. One such approach using partition elements 130 is depicted in FIG. 21.

As suggested previously, there may be a benefit to having device 10 held in place. In FIG. 22, magnetic elements 134 are shown in opposing location in each branch of insertion portion 30. In this embodiment, the two branches of insertion portion 30 are contemplated to be made from a flexible material, such that once the branches are inserted into the two nostrils, the magnets will attract each other, thereby pinching the nasal septum and holding the device in the nose for the duration of the treatment.

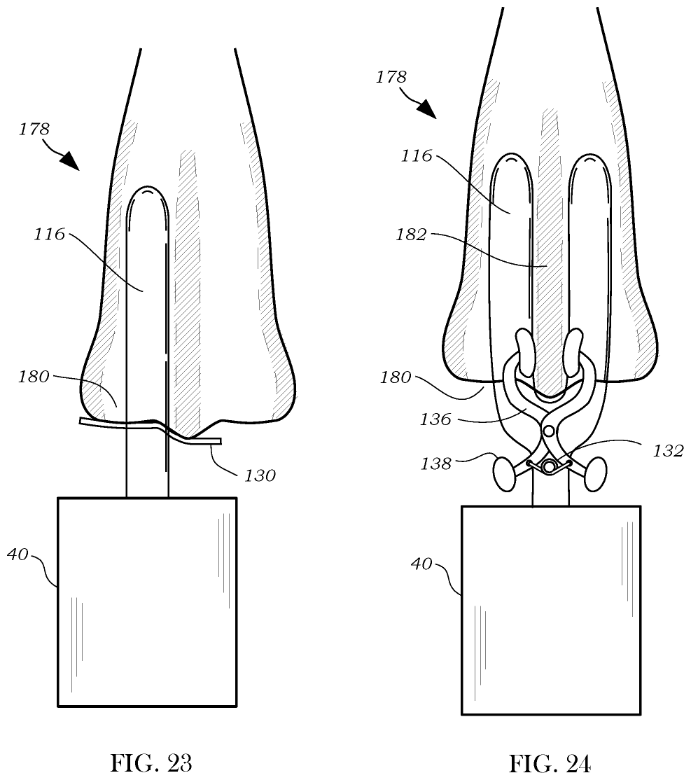

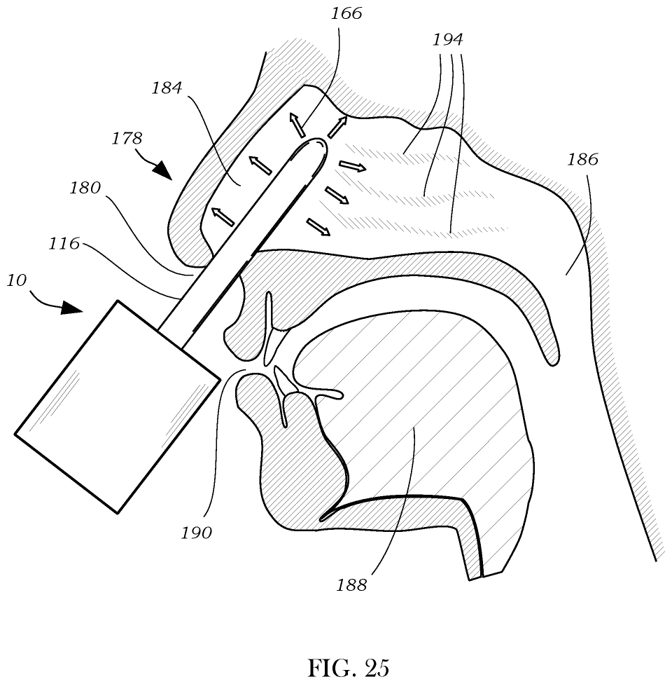

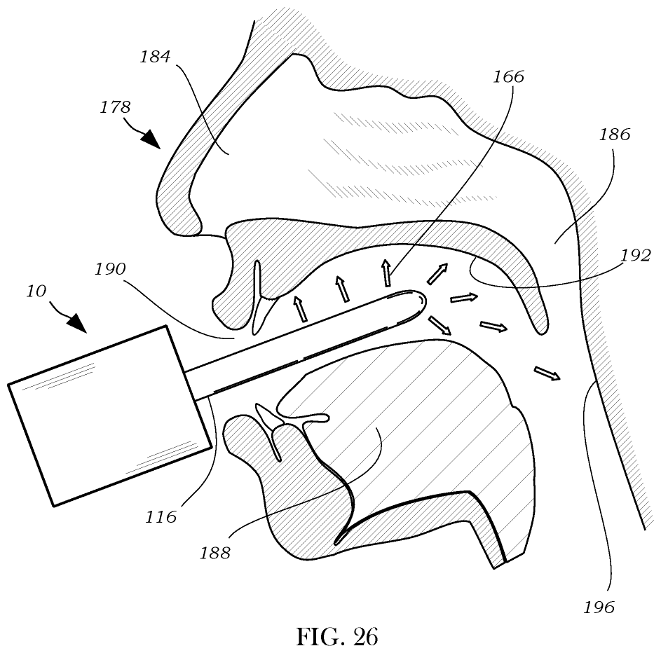

FIG. 23 shows a nose 178 in partial cutaway with the embodiment of FIG. 15 inserted into one nostril opening 180. Partition 130 is shown complying to the curves of the peri-nostril area in order to create a light seal. FIG. 24 shows the embodiment of FIG. 20 inserted into both nostril openings 180. This embodiment has the further feature of a clamp 136 that pinches the nasal septum 182, thereby holding the device in place. The clamping force for clamp 136 is set by spring 132. To release the clamp, the user squeezes clamp release pads 138 together, causing the clamp to open up.

FIG. 25 depicts a section view of a human nose 178 and nasal cavity with a simplified device 10 inserted through a nostril opening 180 into nasal vestibule 184. Light rays 166 show the path of light emitting from light guide 116 into nasal vestibule 184 and preferably beyond, reaching the nasal conchae 194 and nasopharynx 186.

An alternative use for device 10 is for oral disinfection, to inactivate bacteria, fungus, viruses and other pathogens. FIG. 26 shows a simplified device 10 inserted into mouth 190. As shown, light rays 166 preferably reach the inner surfaces of the oral cavity including the soft palate 192, tongue 188 and pharyngeal wall 196.

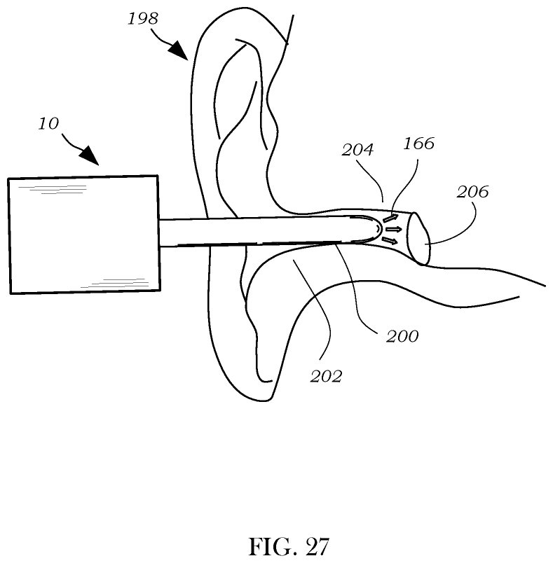

Yet another use for the invention is shown in FIG. 27, wherein a simplified device 10 is shown inserted into the auditory canal 200 of ear 198. Using this method, infections of the outer ear 202 as well as middle ear 204 may be treated. Light rays 166 are shown reaching the ear drum 206 and surrounding areas, and it is understood that the walls of the auditory canal 200 will also be irradiated.

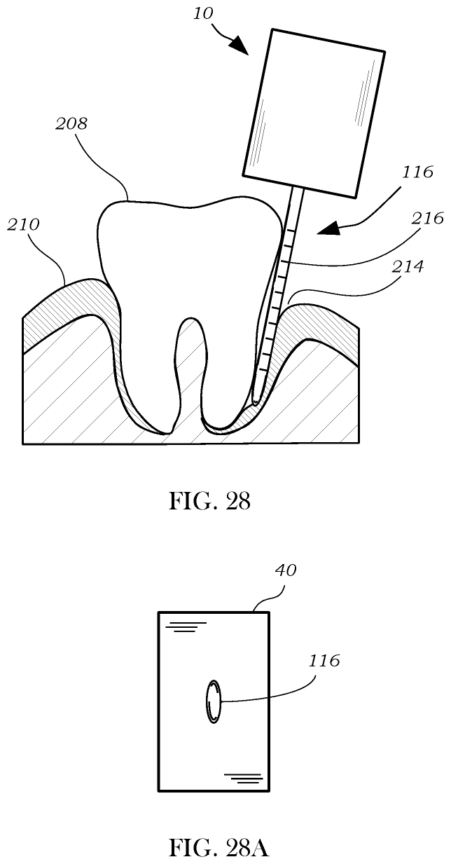

A further embodiment of the present invention is portrayed in FIG. 28. In this embodiment, light guide 116 is thin and may be round or flat, as seen in the front view of FIG. 28A. The application for this embodiment is for treating and optionally diagnosing gum disease surrounding teeth. It is common practice for dental practitioners to use a periodontal probe to measure the depth of the gap or tooth pocket 214 between the side of a tooth 208 and the adjacent gum tissue 210. A deeper pocket is an indication of gum disease, which is typically caused by a bacterial infection and the formation of plaque. Periodontal infections most often involve anaerobes such as Treponema denticola and Porphyromonas gingivalis. The microaerophile Actinobacillus actinomycetemcomitans causes a rare form known as localized juvenile periodontitis. UVC is known to be effective against bacterial species such as these (see Yin et al, “Light based anti-infectives: ultraviolet C irradiation, photodynamic therapy, blue light, and beyond” Curr Opin Pharmacol. 2013 October; 13(5): 10.1016, incorporated herein by reference). In the embodiment of FIG. 28, insertion portion emits UV light, preferably in the range of 200 nm to 230 nm, but not limited thereto. Optionally, depth indications 216 along the length of light guide 116 indicate how deep the tooth pocket 214 is when insertion portion 216 is inserted. In the preferred method, when the depth indications exceed a certain threshold, suggesting gum disease, the dental practitioner can enable the device 10 to emit UV light for a certain period of time to kill the bacteria that may be causing the gum disease.

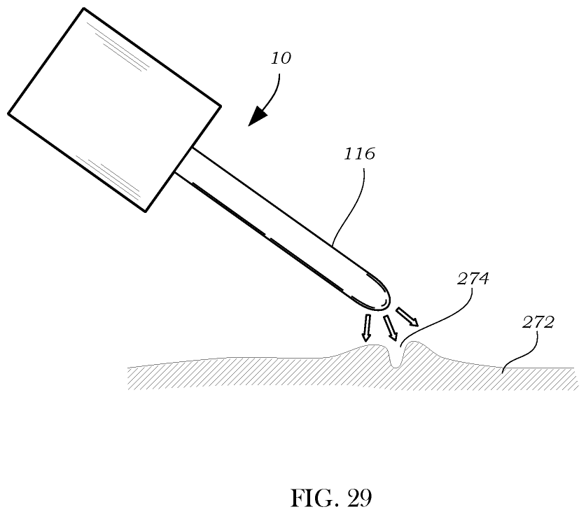

FIG. 29 shows an embodiment of device 10 configured to treat skin lesions, wounds, ulcers, acne and the like. As an example, a skin wound 274 may be infected with staphylococci or streptococci, and may even be infected with an antibioticresistant strain such as MRSA. Far-UV light has been shown to be effective against such infections (Ponnaiya et al, “Far-UVC Light Prevents MRSA Infection of Superficial Wounds in Vivo”, PLoS One 2018 Feb. 21; 13(2):e0192053). Bacterial skin infections are dramatically more problematic in immune-compromised patients (cancer or HIV-infected), and typically more clinically impactful, ranging from impetigo to folliculitis and cutaneous abscess infections. Toenail fungal infections, canker sores, infected insect bites and stings, and decubitis and diabetic foot ulcers may also be treated with device 10. As shown in FIG. 29, device 10 is configured to emit UV light rays 166 to a targeted location, such as skin lesion 274. It will be appreciated that for treating cutaneous lesions, the light guide 116 can be designed to optimize the amount of light emitted from the tip. For example, a reflective coating could be applied along the length of light guide 116, so that the preponderance of light energy is emitted from the tip region. Alternatively, the light guide 116 could be a hollow metal tube with a UV-reflective inner surface, and the tip section could be fused silica. Other application-specific light guide and tip designs can be configured.

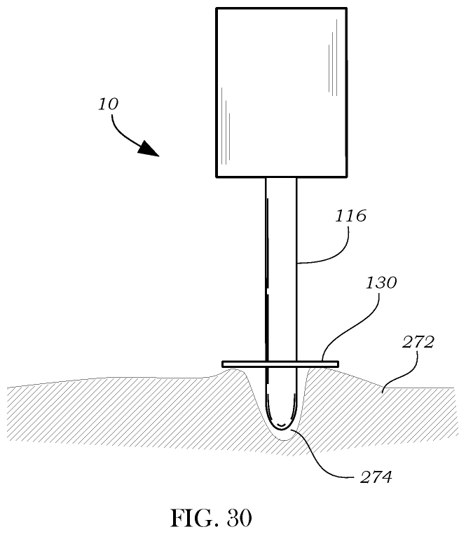

FIG. 30 depicts another application for device 10 wherein an opening or cavity 276 is shown. Opening or cavity 276 may be a deep cut or ulceration, a surgical wound, or the like. The tip of light guide 116 is inserted into the opening or cavity 276 and partition 130 is positioned adjacent to the opening or cavity 276. UV light is then applied and partition 130 confines the UV light to the treatment area. Partition 130 may take many forms besides the disk shape and bulbous forms previously disclosed. For example, it may be cone-shaped, hemispherical or box-shaped, and may be tailored to provide the best protection for a specific treatment site. It will be appreciated that this method and configuration may be applied not only to skin lesions, cuts, ulcers, surgical wounds and the nasal and oral cavities, but also to other anatomical locations where pathogens may invade, such as the conjunctiva, vagina, finger and toenails and the like. Further, the methods and apparatus disclosed herein may be applied to veterinary treatment as well.

Although this invention has been disclosed in the context of a certain preferred embodiment, it will be understood by those skilled in the art that the present invention extends beyond the specifically disclosed embodiment to other alternative embodiments and/or uses of the invention and obvious modifications and equivalents thereof. In particular, while the present optical therapy devices, systems and methods have been described in the context of a particularly preferred embodiment, the skilled artisan will appreciate, in view of the present disclosure, that certain advantages, features and aspects of optical therapy devices, systems and methods may be realized in a variety of other combinations and embodiments. Additionally, it is contemplated that various aspects and features of the invention described can be practiced separately, combined together, or substituted for one another, and that a variety of combination and Subcombinations of the features and aspects can be made and still fall within the scope of the invention. Thus, it is intended that the scope of the present invention herein disclosed should not be limited by the particular disclosed embodiments described above, but should be determined only by a fair reading of the claims that follow.

1. A method of inactivating pathogens in a mammalian body cavity using an ultraviolet (UV) light emitter, including the steps of:
connecting the emitter to an energy source, causing the emitter to emit ultraviolet light,
directing the light into the body cavity,
wherein the bandwidth of UV light includes between about 200 nm and about 230 nm

2. The method of claim 1 wherein the emitter comprises at least one LED.

3. The method of claim 2 wherein the at least one LED emits UV energy centered at about 222 nm.

4. The method of claim 2 wherein the at least one LED is fabricated using aluminum gallium nitride.

5. The method of claim 1 wherein the emitter comprises an excimer light source.

6. The method of claim 5 wherein the excimer light source is a microplasma array.

7. The method of claim 5 wherein the working excimer molecule is predominantly KrBr.

8. The method of claim 5 wherein the working excimer molecule is predominantly KrCl.

9. The method of claim 1 wherein the emitter comprises an LED or laser diode emitting between about 400 nm and 445 nm combined with a β-Barium Borate nonlinear crystal in a resonant cavity to produce the UV.

10. The method of claim 1 wherein the emitter comprises a xenon flash lamp in combination with a bandpass filter.

11. The method of claim 1 wherein the body cavity is the nasopharyngeal or oropharyngeal passageway.

12. The method of claim 1 wherein the body cavity is the auditory canal, outer ear, middle ear or ear drum.

13. The method of claim 1 further including the step of blocking substantially all the UV light from escaping from the cavity by means of positioning a partitioning element adjacent to the opening of the cavity.

14. The method of claim 1 further including the step of sensing the level of ambient visible light and disabling the UV light if the ambient visible light exceeds a pre-determined threshold.

15. The method of claim 1 further including a light guide to direct the UV into the body cavity and a cap to cover and protect the light guide when not in use, including the step of self-disinfecting the light guide and cap using the UV light when the cap is placed over the light guide.

16. A method of inactivating viruses without substantially destroying the microbiome in a mammalian body cavity using an ultraviolet light emitter, including the steps of:
connecting the emitter to an energy source, causing the emitter to emit ultraviolet light between about 200 nm and about 230 nm,
directing the light onto target surfaces of the body cavity.

17. The method of claim 16 wherein the radiant exposure levels of the target surfaces is between 0.1 mJ/cm2 and 10 mJ/cm2.

18. The method of claim 16 wherein the radiant exposure levels of the target surfaces is between 1 mJ/cm2 and 3 mJ/cm2.

19. The method of claim 16 wherein the emitter comprises at least one LED.

20. The method of claim 19 wherein the at least one LED emits UV energy centered at about 222 nm.

21. The method of claim 19 wherein the at least one LED is fabricated using aluminum gallium nitride.

22. The method of claim 16 wherein the emitter comprises an excimer light source.

23. The method of claim 22 wherein the excimer light source is a microplasma array.

24. The method of claim 22 wherein the working excimer molecule is predominantly KrBr.

25. The method of claim 22 wherein the working excimer molecule is predominantly KrCl.

26. The method of claim 16 wherein the emitter comprises an LED or laser diode emitting between about 400 nm and 445 nm combined with a β-Barium Borate nonlinear crystal in a resonant cavity to produce the UV.

27. A method of treating gingivitis with a device having a UV emitter emitting light between about 200 nm and 230 nm and an insertion portion, comprising the steps of:
inserting the insertion portion at least partially into the space between a tooth and the surrounding gum tissue, and
applying UV to the surfaces of the tooth and surrounding gum tissue.

28. The method of claim 27 further comprising the step of measuring the depth of insertion of the insertion portion into the space between the tooth and surrounding gum tissue by reading depth indications marked on the insertion portion.

29. A method of disinfecting a treatment area having a bacterial or fungal skin infection, ulcer, canker sore, laceration or surgical wound with a device having a UV emitter emitting light between about 200 nm and 230 nm and a light guide, comprising the steps of:
positioning the light guide adjacent to the treatment area, and
applying UV to the treatment area for a period of time.

30. The method of claim 29 further comprising the step of surrounding the treatment area with a partitioning element to substantially block UV from escaping from the treatment area.
